(19) United States
(12) Patent Application Publication
(10) Pub. No.: US 2026/0232207 A1
(43) Pub. Date: Aug. 13, 2026

(54) SYSTEM AND METHOD FOR MEASURING CHARACTERISTICS OF CORONARY ARTERIAL SYSTEM

(71) Applicant: Stallion Cardio, LLC, Happy Valley, OR (US)

(72) Inventor: Yohan Chacko, Graceville (AU)

(21) Appl. No.: 19/152,891

(22) PCT Filed: Jan. 30, 2024
(86) PCT No.: PCT/US2024/013521

§ 371 Date: Jul. 31, 2025

Related U.S. Application Data
(60) Provisional application No. 63/442,705, filed on Feb. 1, 2023.

(51) Int. Cl. A61B 5/0215; A61B 5/00; A61B 5/02; A61B 5/026

(52) U.S. Cl. CPC A61B 5/0215; A61B 5/02007; A61B 5/026; A61B 5/6851; A61B 5/6852; A61B 2562/0247

(57) ABSTRACT

Disclosed herein is a method for evaluating a blood vessel of a patient. The method can include equalizing pressure measured by a guide wire sensor to pressure measured by a catheter sensor when the guide wire sensor is positioned within the blood vessel and distal to a stenosis of the blood vessel and when the catheter sensor is positioned within the blood vessel and proximal to the stenosis. Subsequent to equalizing, the guide wire sensor may be retracted so that the guide wire sensor and the catheter sensor are both positioned proximal to the stenosis, and then pressure may be measured by the guide wire sensor. Pressure measured by the guide wire sensor can be used to generate an indication that is indicative of a characteristic of the stenosis. The indication can be output for presentation to a clinician to permit the clinician to assess the stenosis.

### BACKGROUND

Fractional Flow Reserve (FFR), Instantaneous wave-free ratio (iFR), and other hyperemic/non-hyperemic ratios are calculated indices that clinicians may use for assessing a blood vessel of a patient. Traditional approaches for determining these indices have suffered from problems such as the calibration of sensor measurements shifting over time (sometimes referred to as drift) and the method of collecting said measurements being cumbersome and time consuming. The result can be reduced attempts by physicians to appropriately gather the measurements, as well as inaccuracies in sensor measurements used to generate the indices. Ultimately, non-existent or inaccurate indices can result in misdiagnosing the condition of a blood vessel, which can have grave consequences for the patient.

To determine such indices, traditionally a guide wire sensor is first equalized to a catheter sensor when both the guide wire sensor and the catheter sensor are positioned proximal to the stenosis (i.e., proximal to the stenosis may refer to a position upstream from the stenosis relative to a blood flow in the blood vessel) and once a guide wire is exited a catheter but in a retracted position in the aorta or disease-free ostium (e.g., beginning portion) of the coronary artery. The guide wire sensor is then extended to be positioned distal to the stenosis (i.e., distal to the stenosis may refer to a position downstream from the stenosis relative to a blood flow in the blood vessel), and then pressure is measured by the guide wire sensor. To determine whether the guide wire sensor experienced a shift in sensor calibration between the equalization and the determination of pressure measurement when the guide wire sensor is positioned distal to the stenosis, the guide wire sensor is next retracted to be positioned proximal to the stenosis. Then, pressure measured by the guide wire sensor and the catheter sensor are compared to check for equalization between the guide wire sensor and the catheter sensor. If the check indicates no shift in sensor calibration and loss of equalization, the pressure measured when the guide wire sensor is positioned distal to the stenosis is used to determine a hyperemic or non-hyperemic ratio, such as FFR or iFR.

### SUMMARY

Features disclosed herein may address issues with traditional approaches for assessing a blood vessel. The features can, for example, enable determination of an index that may be used similarly to or in place of FFR, iFR, and other non-hyperemic ratios and which may be significantly less susceptible to a shift in sensor calibration. The features desirably may facilitate relatively fast computing of the index, obviating of a traditional step of checking sensor calibration, reducing an amount of time for taking sensor measurements and during which a sensor is positioned within a blood vessel, and providing a reliable indication of a characteristic of the stenosis for a clinician.

Disclosed is at least a method for evaluating a blood vessel of a patient. The method can include equalizing pressure measured by a guide wire sensor to pressure measured by a catheter sensor when the guide wire sensor is positioned within the blood vessel and distal to a stenosis of the blood vessel and when the catheter sensor is positioned within the blood vessel and proximal to the stenosis. Subsequent to equalizing, the guide wire sensor may be retracted so that the guide wire sensor and the catheter sensor are positioned proximal to the stenosis, and then pressure may be measured by the guide wire sensor and the catheter sensor. Pressure measured by the guide wire sensor and the catheter sensor can be used to generate an indication (for example, a ratio of pressure measured by the guide wire sensor and the catheter sensor when the guide wire sensor and the catheter sensor are positioned proximal to the stenosis) that is indicative of a characteristic of the stenosis (for example, a size or a flow reduction caused by the stenosis). In turn, the indication can be output for presentation, such as on a display, to a clinician to permit the clinician to assess the stenosis. The method can be used to determine hyperemic or non-hyperemic measurements of pressure.

Equalizing the pressure measured by the guide wire sensor to the pressure measured by the catheter sensor when the guide wire sensor is positioned distal to the stenosis and the catheter sensor is positioned proximal to the stenosis can be counterintuitive. Such timing for equalizing likely results in the guide wire sensor and the catheter sensor being equalized to one another when the guide wire sensor and the catheter sensor are in different pressure environments. The guide wire sensor is positioned distal to the stenosis where pressure is likely less than pressure proximal to the stenosis. A clinician would not traditionally seek to equalize the guide wire sensor to the catheter sensor when the guide wire sensor and the catheter sensor are in different pressure environments because the guide wire sensor and the catheter sensor would be believed to be subjected to “unequal” conditions and thus unsuitable for equalizing to one another. A clinician may consider such timing for equalizing to result in a miscalibration of pressure measurements rather than being able to facilitate any determination of useful information.

It was discovered, however, that such timing for equalizing (i.e., when the guide wire sensor is positioned distal to the stenosis and the catheter sensor is positioned proximal to the stenosis) can permit a safer and faster assessment of the stenosis. Such timing may significantly diminish (if not entirely reduce) any shift in sensor calibration during the measurement process at least because retracting a guide wire with its guide wire sensor is a safer, faster, and easier process than extending the guide wire. Retracting the guidewire involves relatively quickly pulling the guide wire back through the blood vessel while extending the guide wire involves traversing the guide wire forward generally along the middle of the blood vessel and through turns, narrowing, or blockages without traumatizing the blood vessel (for example, tearing the blood vessel with a tip of the guide wire or even causing the tip of the guide wire to puncture and pass outside the blood vessel). Such timing can be less likely to result in a shift in sensor calibration due at least to the faster measurement process which may diminish the chance for any shift in sensor calibration to occur. This desirably can obviate the demand for checking for a shift in sensor calibration or potentially taking repeat measurements if the shift is detected, thereby reducing a number of steps performed. In addition, such timing can facilitate reducing the number of traditional measurement steps, including multiple introducer removals or flushing of the catheter. It was experimentally determined that such timing may speed up the assessment of a stenosis by around 20% to 30% relative to the traditional approaches discussed in the background.

Moreover, the features disclosed herein can enable determination of a measurement of a characteristic (for example, a flow or resistance) of one or more blood vessels where the measurement may be significantly less susceptible to be inaccurate due to a shift in sensor calibration than a traditional measurement of that characteristic. The characteristic may, for instance, be useful for assessing a microvasculature of a patient (for example, for microvascular disease or for blockages in pre-arterioles or arterioles rather than an epicardial vessel) that may not be visible on an angiogram. Very slight changes in the measurements can lead to different recommended treatment pathways, so clinicians rely on hyper accurate measurements and indices calculated from said measurements. The features disclosed herein desirably can facilitate relatively fast computing of the measurements, obviating of a traditional step of checking sensor calibration, reducing an amount of time for taking sensor measurements and during which a sensor is positioned within the one or more blood vessels, and providing reliable indications of one or multiple characteristics of the one or more blood vessels for the clinician.

### BRIEF DESCRIPTION OF THE DRAWINGS

FIG. 1 illustrates a system for assessing a stenosis of a blood vessel of a patient.

FIGS. 2A and 2B illustrate a catheter and a guide wire positioned in a blood vessel for determining pressure distal or proximal to a stenosis of the blood vessel.

FIG. 3 illustrates a process for assessing a stenosis of a blood vessel.

FIG. 4A is a plot of an index versus a ratio of distal pressure (Pd) to aortic pressure (Pa) where the index is determined at least in part using the process of FIG. 3.

FIG. 4B is a plot of an index versus FFR where the index is determined at least in part using the process of FIG. 3.

FIG. 5 illustrates a process for assessing a resistance or other characteristic of a blood vessel.

### DETAILED DESCRIPTION

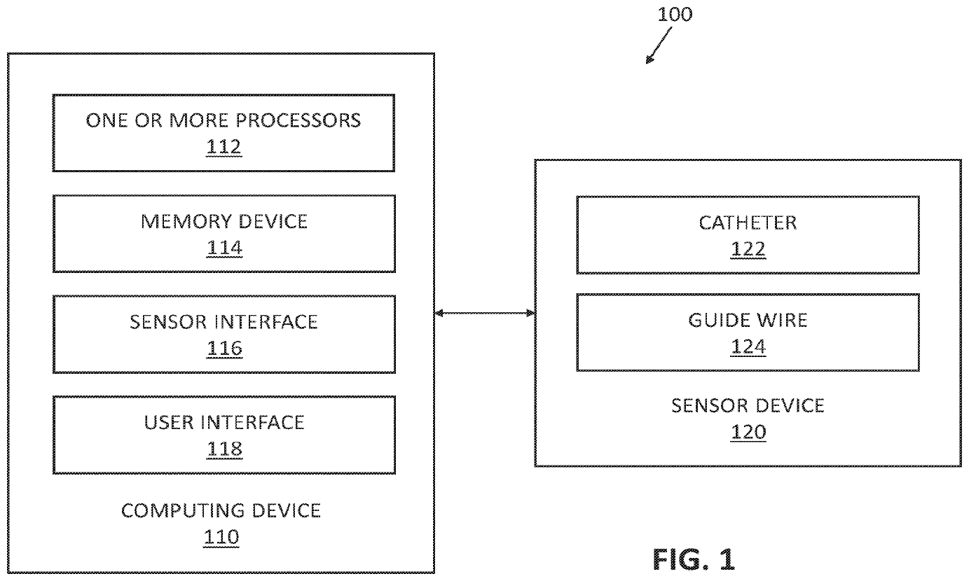

FIG. 1 illustrates a system 100 for assessing a stenosis, such as a coronary lesion, of a blood vessel of a patient. The system 100 can include a computing device 110 and a sensor device 120. The computing device 110 can include one or more processors 112, a memory device 114, a sensor interface 116, and a user interface 118. The sensor device 120 can include a catheter 122 and a guide wire 124 (sometimes referred to as a pressure wire). The computing device 110 may be a patient monitor configured to implement the functionality disclosed herein based on sensing performed by the catheter 122 and the guide wire 124.

The one or more processors 112 can be programmed to execute instructions stored to the memory device 114. The instructions may cause the one or more processors 112 to perform one or more data acquisition, data processing, instrument control, user interface control, or other processing or control operations, such as with the memory device 114, the sensor interface 116, and the user interface 118.

The sensor interface 116 can facilitate communication with one or more sensors of the sensor device 120, such as a pressure sensor of the catheter 122 and a pressure sensor of the guide wire 124. The sensor interface 116 may control one or more operations by the sensor device 120 or receive data collected by the one or more sensors of the sensor device 120.

The user interface 118 can include one or more elements for receiving user inputs or providing user outputs. The one or more elements that receive user inputs can include buttons, switches, dials, touch screens, or the like. The one or more elements that receive user inputs can, for example, receive inputs like a request to equalize the catheter 122 and the guide wire 124, such as pressure measured by a sensor of the guide wire 124 to pressure measured by a sensor of the catheter 122. The one or more elements that provide user outputs can include visual feedback devices (for example, a display or light emitting diodes), haptic feedback devices, or audio devices (for example, speakers), or the like. The one or more elements that provide user outputs can, for example, convey information to the user like an indication determined from pressure measurements by the sensor device 120 or status information like whether the computing device 110 or the sensor device 120 are functioning within desired operating parameters.

The catheter 122 can include a shaft through which the guide wire 124 extends. The catheter 122 and the guide wire 124 can each include one or more sensors, such as a transducer, a piezo-resistive sensor, a piezo-electric sensor, a capacitive sensor, a thermistor, an electromagnetic sensor, a fluid column, an optical sensor, or other monitoring elements, configured to obtain diagnostic information about the blood vessel. The diagnostic information can include one or more of pressure, flow (velocity), images, or temperature. The one or more sensors may be positioned at various locations along the catheter 122 and the guide wire 124, such as at or proximate to distal ends of the catheter 122 and the guide wire 124 or other positions away from the distal ends.

The guide wire 124 can extend and retract relative to the catheter 122 when the catheter 122 and the guide wire 124 are positioned in the blood vessel. The guide wire 124 may be in a retracted position where the catheter 122 and the guide wire 124 are positioned proximal to the stenosis. As used herein, proximal to the stenosis may be understood to refer to upstream from the stenosis relative to a blood flow in the blood vessel. The guide wire 124 may alternatively be in an extended position where the catheter 122 remains positioned proximal to the stenosis and the guide wire 124 is extended past the stenosis and positioned distal to the stenosis. As used herein, distal to the stenosis may be understood to refer to downstream from the stenosis relative to the blood flow in the blood vessel.

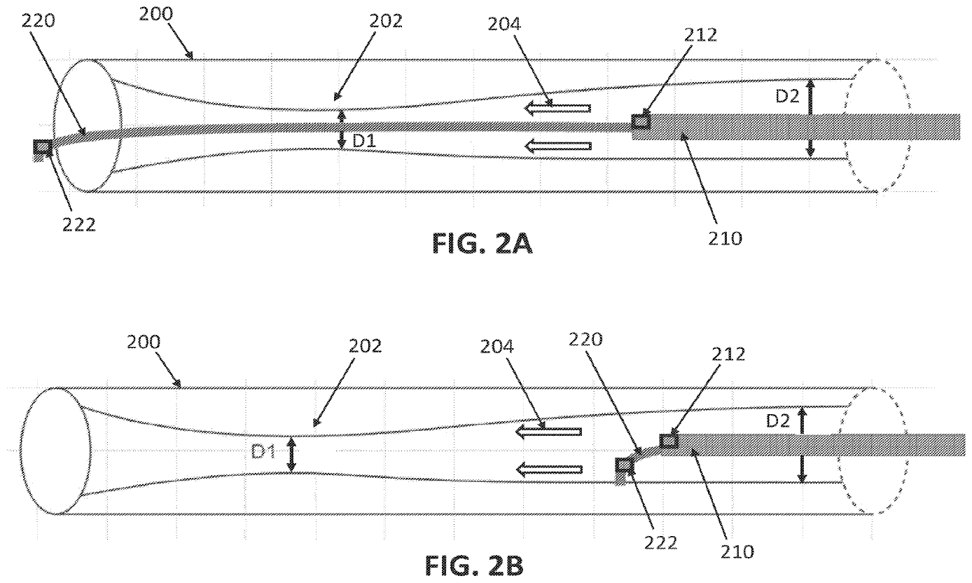

FIG. 2A illustrates a catheter, such as the catheter 122 of FIG. 1, and a guide wire, such as the guide wire 124, positioned in a blood vessel 200 of a patient. The catheter and the guide wire can be used to determine pressure distal or proximal to a stenosis 202 of the blood vessel 200. The stenosis 202 may be a narrowing of the blood vessel 200 where the blood vessel 200 has a diameter D1. The diameter D1 can be less than a diameter D2 of the blood vessel 200 at another location in the blood vessel 200. The direction of blow flow in the blood vessel 200 is depicted by arrows 204. The catheter can include a shaft 210 and a catheter sensor 212 attached to the shaft 210. The catheter sensor 212 may measure pressure, temperature, optical information, or flow in the blood vessel at or proximate to a distal end of the shaft 210, such as at a location within 50, 40, 30, 20, 10, or 5 mm of the distal end. The guide wire can include a wire 220 and a guide wire sensor 222 attached to the wire 220. The guide wire sensor 222 may measure pressure, temperature, optical information, or flow in the blood vessel at or proximate to a distal end of the wire 220, such as at a location within 50, 40, 30, 20, 10, or 5 mm of the distal end.

FIG. 2A shows the wire 220 in an extended position where the catheter sensor 212 is positioned proximal to the stenosis 202 and the guide wire sensor 222 is positioned distal to the stenosis 202. On the other hand, FIG. 2B illustrates the catheter and the guide wire of FIG. 2A positioned in the blood vessel 200 with the wire 220 in a retracted position where the catheter sensor 212 and the guide wire sensor 222 are positioned proximal to the stenosis 202.

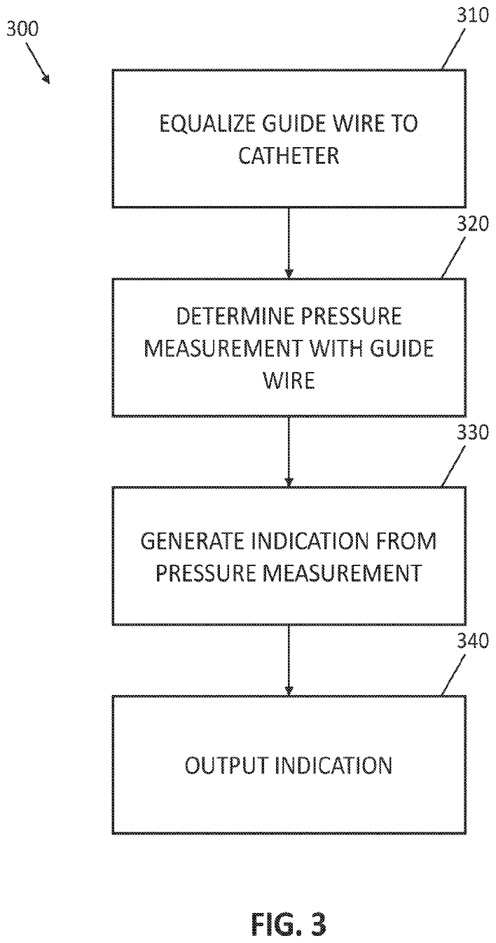

FIG. 3 illustrates a process 300 for assessing a stenosis of a blood vessel of a patient. The process 300 can be performed, for instance, by the computing device 110 of FIG. 1 using the sensor device 120 of FIG. 1, which could include the shaft 210, the catheter sensor 212, the wire 220, and the guide wire sensor 222 of FIGS. 2A and 2B. The process 300 can advantageously, in certain implementations, provide an approach for more safely and quickly assessing the stenosis than other approaches. This can be at least because the process 300 may utilize a counterintuitive timing for equalization that permits faster collection of sensor measurements than the other approaches and obviates the demand to perform a checking step to confirm whether sensor calibration undesirably drifted over time. The process 300 can be used to determine hyperemic or non-hyperemic measurements of pressure.

At block 310, the process 300 can equalize a guide wire to a catheter. For example, the one or more processors 112 can equalize a pressure measured by the guide wire 124, such as with the guide wire sensor 222, to a pressure measured by the catheter 122, such as with the catheter sensor 212. The equalizing can include one or more of (i) matching a mean pressure of a pressure waveform measured by the guide wire sensor 222 to a mean pressure of a pressure waveform measured by the catheter sensor 212, (ii) matching a timing of the pressure waveform measured by the guide wire sensor 222 to a timing of the pressure waveform measured by the catheter sensor 212, (iii) matching a gain of the pressure waveform measured by the guide wire sensor 222 to a level of the pressure waveform measured by the catheter sensor 212, (iv) matching an oscillator frequency of the pressure waveform measured by the guide wire sensor 222 to a frequency of the pressure waveform measured by the catheter sensor 212, or (v) matching a damping factor of the pressure waveform measured by the guide wire sensor 222 to a damping of the pressure waveform measured by the catheter sensor 212. The equalizing may be performed responsive to an input from a user, such as via the user interface 118. The input may, for example, indicate to initiate a pressure measurement or to equalize the guide wire sensor 222 and the catheter sensor 212 (such as by the user selecting an equalize button on the user interface 118). Equalization may, in some instances, be referred to as normalization or nullification.

Block 310 may be performed when the shaft 210 and the wire 220 are positioned within the blood vessel 200 and the catheter sensor 212 is positioned proximal to the stenosis and the guide wire sensor 222 is positioned distal to the stenosis, as illustrated in FIG. 2A. Such timing for performing equalization can result in the catheter sensor 212 and the guide wire sensor 222 equalizing in different pressure environments at least because the guide wire sensor 222 is positioned distal of the stenosis where pressure may be less than pressure proximal of the stenosis where the catheter sensor 212 is positioned. This timing for equalization is counterintuitive because a clinician would not traditionally seek to equalize the guide wire sensor 222 to the catheter sensor 212 when the guide wire sensor 222 and the catheter sensor 212 are known to be in different pressure environments. Such timing would be expected to result in a miscalibration of pressure measurements.

At block 320, the process 300 can determine a pressure measurement with the guide wire. For example, the one or more processors 112 can determine a pressure measurement using guide wire 124, such as with the guide wire sensor 222. Block 320 may be performed when the guide wire sensor 222 and the catheter sensor 212 are both within the blood vessel and positioned proximal to the stenosis, as illustrated in FIG. 2B. Block 320 can be performed subsequent to equalizing at block 310 and without again equalizing the guide wire 124 to the catheter 122. Block 320 may be performed while the guide wire sensor 222 is in motion, such as once the guide wire sensor 222 enters or entirely passes through the stenosis, or once the guide wire sensor 222 is at rest, such as when the wire 220 is in a fully retracted position. The process 300 may also at block 320 determine a pressure measurement with the catheter 122, such as with the catheter sensor 212.

Block 320 may be performed within a short period of time, such as within 30, 20, 15, 10, 5, 4, 3, or 2 seconds (for example, within around 3-10 seconds when it is desired to minimize a delay between block 320 and block 310), subsequent to equalizing at block 310. Block 320 can be performed within the short time period at least because the wire 220 may be moved relatively quickly and safely from the extended position to the retracted position. In contrast, moving the wire 220 from the retracted position to the extended position may be a relatively slower, less safe, and more difficult operation to perform because the extending results in the wire 220 progressing farther into the blood vessel 200 generally along the middle of the blood vessel 200 and potentially traversing changes in path direction (including at least changes in tortuosity) and various obstacles (including at least the stenosis) without traumatizing the blood vessel 200 (for example, tearing the blood vessel 200 with a tip of the wire 220 or even causing the tip of the wire 220 to puncture and pass outside the blood vessel 200).

At block 330, the process 300 can generate an indication from the pressure measurement. For example, the one or more processors 112 can generate an indication from the pressure measurement determined at block 320 using the guide wire 124 when the guide wire sensor 222 was positioned proximal to the stenosis. The indication can be a value indicative of one or more of a pressure change around the stenosis, a resistance caused by the stenosis, or a rate of blood flow through the stenosis. The generating can, for instance, include determining a pressure ratio or a pressure difference (sometimes referred to as a pressure gradient) from (i) the pressure measurement from guide wire sensor 222 when the guide wire sensor 222 was positioned proximal to the stenosis and (ii) a pressure measurement from the catheter sensor 212 when the catheter sensor 212 was positioned proximal to the stenosis. The generating may be performed for each heartbeat cycle, over multiple heartbeat cycles, or for some individual heartbeat cycles (or portions thereof) but not others. The pressure measurements from the guide wire sensor 222 and the catheter sensor 212 can each be a mean, median, mode, or other suitable value determined from a detected pressure waveform. The pressure measurements from the guide wire sensor 222 and the catheter sensor 212 may each be determined from a particular portion or an entirety of the detected pressure waveform for each heartbeat cycle. The detected pressure waveforms may be conditioned, filtered (such as to remove outliers), or processed prior to being used to determine one or more pressure measurements.

The pressure ratio may equal a ratio of a pressure measurement from guide wire sensor 222 when the guide wire sensor 222 was positioned proximal to the stenosis and a pressure measurement from the catheter sensor 212 when the catheter sensor 212 was positioned proximal to the stenosis. Such a pressure ratio can generally be greater than 1.0 because the guide wire sensor 222 was equalized to the catheter sensor 212 when the guide wire sensor 222 was positioned distal to the stenosis. The guide wire sensor 222 accordingly moved from a lower pressure environment distal to the stenosis to a higher pressure environment proximal to the stenosis, causing the guide wire sensor 222 to register an increase in pressure from the repositioning. In some implementations, a combined waveform may be determined by the one or more processors 112 from dividing and filtering (i) a pressure waveform from guide wire sensor 222 that is detected when the guide wire sensor 222 is positioned proximal to the stenosis and (ii) a pressure waveform from catheter sensor 212 that is detected when the catheter sensor 212 is positioned proximal to the stenosis, and the generating can involve selecting a highest point on the combined waveform to be the pressure ratio (sometimes referred to as an index).

This pressure ratio may contrast from traditional Pd/Pa ratios (where Pd is a pressure measurement from a guide wire sensor sensing a distal coronary pressure and Pa is a pressure measurement from a catheter sensor sensing an aortic pressure) at least because the traditional Pd/Pa ratios may be less than 1.0, because the guide wire sensor is first equalized to the catheter sensor when the guide wire sensor and the catheter sensor are both positioned proximal to a stenosis and then the guide wire sensor is moved from a higher pressure environment proximal to the stenosis to a lower pressure environment distal to the stenosis to measure Pd.

The generating can, in some implementations, involve implementing an algorithm that outputs an index normalized to traditional Pd/Pa ratios (or widely adopted indices), such as by inverting the pressure ratio. The inverted pressure ratio may thus be greater than 1.0 rather than less than 1.0. The inverted pressure ratio may be further adjusted according to the absolute pressure differences in Pd and Pa pressure measurements using this novel approach. The system and methods disclosed herein are accordingly uniquely flexible in that they can be used to either reshape the indices being output to clinicians or can be normalized to become more accurate, reliable versions of existing indices.

In yet other examples, at block 330, the indication can be a value that reflects a resistance caused by the stenosis or a rate of blood flow through the stenosis. Such a value may be determined using approaches similar to those used, for instance, to compute an index of microcirculatory resistance (IMR) or coronary flow reserve (CFR). Inputs used to determine such a value can include (i) temperature measurements by the catheter 122 or the guide wire 124, (ii) optical measurements by the catheter 122 or the guide wire 124, (iii) pressure measurements by the catheter 122 or the guide wire 124, or (iv) a determined resting transit time or a determined hyperemic transit time. Steps taken to determine such a value can include at zeroing pressure of the catheter 122 or the guide wire 124 to atmosphere.

At block 340, the process 300 can output an indication. For example, the one or more processors 112 can output the indication determined at block 330 for presentation to a user via the user interface 118, such as in the form of a numerical value, a plot, an alarm, or a coloring of an object that is presented to the user. The indication may permit the user to assess the stenosis, such as a size or severity of the stenosis or a reduction in blood flow caused by the stenosis. The one or more processors 112 may additionally or alternatively output the indication for storage to the memory device 114, output the indication via wired or wireless communication to an electronic device, or output the indication to automatically activate one or more control operations (for example, to cause delivering of a fluid to the patient, determining a type or quantity of medication appropriate for the patient in view of the pressure ratio, ordering a medication for the patient, determining a size or type of stent for placement in the blood vessel in view of the pressure ratio, placing a stent in the blood vessel, or submit a request for assistance from another clinician in view of the pressure ratio).

The process 300 can include one or more additional steps or may exclude one or more steps in some implementations. For example, the process 300 can include, after block 310 and prior to block 320, periodically (for instance, every 1 ms, 10 ms, or sufficiently frequently so as to appear instantaneous to a user) determining a pressure measurement with the guide wire 124 and outputting the pressure measurement as part of a pressure trace (sometimes referred to as a pullback trace) on a display to a user. The pressure trace may be used by the user to observe pressure changes in the blood vessel while the guide wire 124 retracts and passes by one or more stenosis. The pressure trace can indicate to the user a location in the blood vessel where a relatively large change in pressure occurs and a stent may be placed to reduce that pressure change. In such an implementation, block 320 may be performed within around 30 seconds subsequent to equalizing at block 310. This duration may, for instance, be sufficient to determine the pressure measurements for the pressure trace, as well as generally still minimize a delay between block 320 and block 310. As another example, the process 300 may further include removing an introducer, flushing the catheter, infusing adenosine in the blood vessel, or waiting for hyperemia. As yet another example, although some generated indications or determined values are explained in connection with the process 300, other indications may be generated or values may be determined prior to initiating the process 300, during or between one more steps of the process 300, or after completing the process 300, so as to further improve the efficiency of collecting useful information for a clinician when performing the process 300.

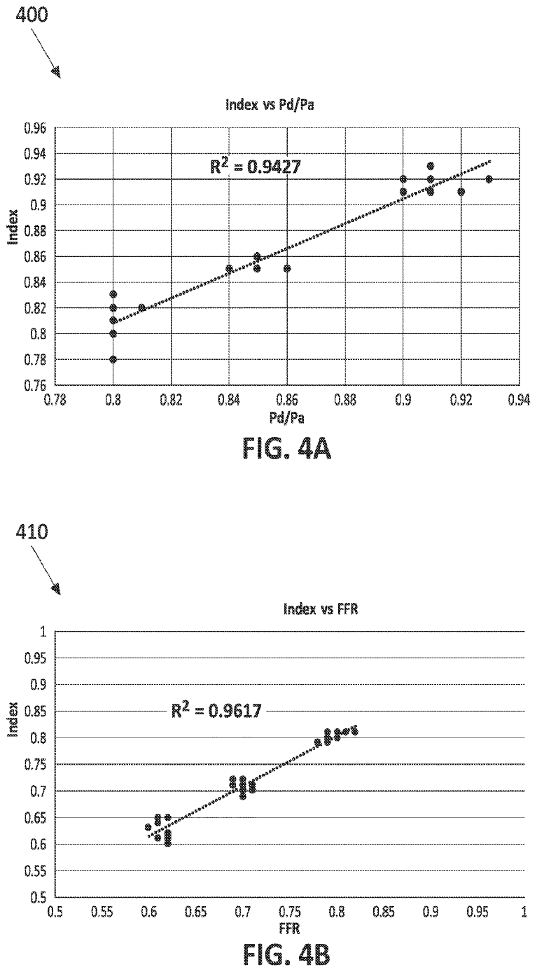

FIG. 4A depicts a plot 400 of an index versus Pd/Pa where the index is determined at least in part from the process 300 of FIG. 3. The index illustrated in the plot 400 may be a reciprocal of the index discussed with respect to the process 300 so that all compared values are less than 1.0. The plot 400 was prepared based on experimentally determined data. The plot 400 illustrates how the process 300 can be used to determine an index that has a relatively high correlation (R²=0.9427) to traditionally determined Pd/Pa. This relatively high correlation demonstrates the clinical significance of the index disclosed herein. Pd/Pa for the plot 400 was determined using the approach of: equalizing a guide wire sensor to a catheter sensor in a blood vessel when the catheter sensor and the guide wire sensor are positioned proximal to a stenosis of a blood vessel, then measuring pressure with the guide wire sensor when the guide wire sensor is positioned distal to the stenosis, next measuring pressure with the catheter sensor and the guide wire sensor when the catheter sensor and the guide wire sensor are positioned proximal to the stenosis to check for sensor calibration drift, and finally determining Pd/Pa from pressure measured when the guide wire sensor was positioned distal to the stenosis and the catheter sensor was positioned proximal to the stenosis.

FIG. 4B depicts a plot 410 of an index versus FFR where the index is determined at least in part from the process 300 of FIG. 3. The index may be a hyperemic index. The index illustrated in the plot 410 may be a reciprocal of the index discussed with respect to the process 300 so that all compared values are less than 1.0. The plot 410 was prepared based on experimentally determined data. The plot 410 illustrates how the process 300 can be used to determine an index that has a relatively high correlation (R²=0.9617) to FFR. This relatively high correlation demonstrates the clinical significance of the index disclosed herein. FFR was determined using the approach of: equalizing a guide wire sensor to a catheter sensor in a blood vessel when the catheter sensor and the guide wire sensor are positioned proximal to a stenosis of a blood vessel, then measuring pressure with the guide wire sensor when the guide wire sensor is positioned distal to the stenosis under hyperemic conditions, next measuring pressure with the catheter sensor and the guide wire sensor when the catheter sensor and the guide wire sensor are positioned proximal to the stenosis to check for sensor calibration drift, and finally determining FFR from pressure measured when the guide wire sensor was positioned distal to the stenosis and the catheter sensor was positioned proximal to the stenosis.

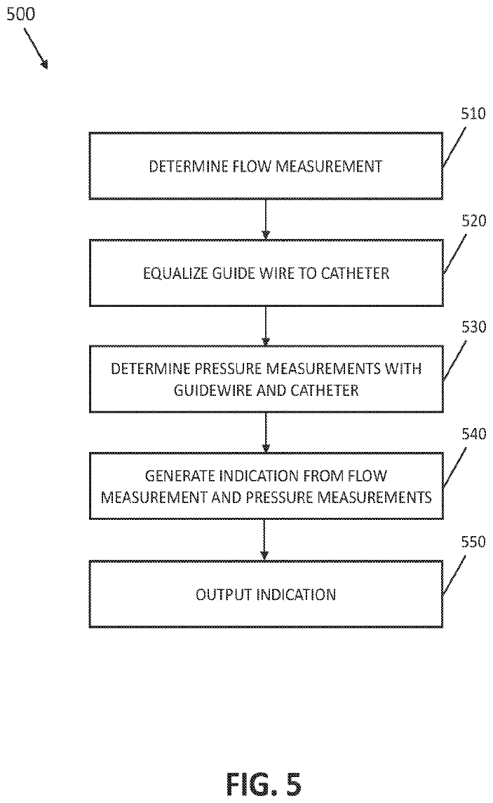

FIG. 5 illustrates a process 500 for assessing a resistance or other characteristic of a blood vessel of a patient. The blood vessel may include a microvasculature of the patient, such as one or more pre-arterioles or arterioles. The process 500 can be performed, for instance, by the computing device 110 of FIG. 1 using the sensor device 120 of FIG. 1, which could include the shaft 210, the catheter sensor 212, the wire 220, and the guide wire sensor 222 of FIGS. 2A and 2B. Although aspects of the process 500 may be described with reference to FIGS. 2A and 2B, the blood vessel 200 may not include the stenosis 202 when the process 500 is discussed in connection with FIGS. 2A and 2B.

The process 500 can advantageously, in certain implementations, provide an approach for more safely, accurately, and quickly assessing the resistance or other characteristic than other approaches. This can be at least because the process 500 may utilize a counterintuitive timing for equalization that permits faster collection of sensor measurements than the other approaches and obviates the demand to perform a checking step to confirm whether sensor calibration undesirably drifted over time. The process 500 may be used by a clinician to assess a patient's microvasculature (such as for a disease or a blockage) that may not be visible on an angiogram. The process 500 (or one or more blocks of the process 500) can be initiated responsive to an input from a user, such as via the user interface 118. The input may, for example, indicate to initiate the process 500 (such as by the user selecting a start button on the user interface 118).

At block 510, the process 500 can determine a flow measurement. For example, the one or more processors 112 can determine a flow measurement using guide wire 124, such as with the guide wire sensor 222, or the catheter 122, such as the catheter sensor 212, when the shaft 210 and the wire 220 are positioned within the blood vessel 200. The guide wire sensor 222 or the catheter sensor 212 may measure a temperature, a blood flow velocity, or a Doppler flow signal in the blood vessel 200 that are usable to determine the flow measurement.

The guide wire 124 can at block 510 be in an extended position relative to the catheter 122, meaning the wire 220 is in an extended position relative to the shaft 210. The guide wire 124 may extend from the catheter 122 in a direction of a blood flow in the blood vessel 200. The guide wire sensor 222 may be positioned distal to a stenosis or another structure of the blood vessel 200 (for example, a fork or turn in a path of the blood vessel 200) when the guide wire 124 is in the extended position relative to the catheter 122, or the guide wire sensor 222 may not be positioned distal to such a structure of the blood vessel 200 when the guide wire 124 is in the extended position relative to the catheter 122. The guide wire 124 can extend into a proximal, mid, or distal segment of the coronary artery when in the extended position relative to the catheter 122. The guide wire sensor 222 can be separated from the catheter sensor 212 in the blood vessel 200 by a distance (for example, by at least 50, 60, 70, 75, 80, 90, 100, 110, 120, 125, 130, 140, 150, 160, 170, 175, 180, 190, 200, 210, 220, 225, 230, 240, or 250 mm) when the guide wire 124 is in the extended position relative to the catheter 122. For instance, the guide wire 124 may extend around 160 mm relative to the catheter 122 when the guide wire 124 is extended in a coronary artery (such as a left anterior descending artery, a right coronary artery, or a left circumflex artery).

The one or more processors 112 may, for instance, determine the flow measurement according to the following thermodilution procedure: One or more resting and hyperemic transit time measurements (which may directly correlate with blood flow because a high flow results in cooling more quickly than a low flow) can be determined by comparing the time it takes for the blood in the blood vessel 200 to return to a normal body temperature (for example, 37° C.) after an injection (for example, 3 mL of 20-25° C. saline injected in artery) at rest and with hyperemia (for example, after delivery of adenosine, which vasodilates microcirculation and simulates stress situation causing a maximal blood flow). The time for the blood in the blood vessel 200 to return to the normal body temperature may be determined using multiple temperature measurements from the guide wire sensor 222 at rest. The time for the blood in the blood vessel 200 to return to the normal body temperature at rest may be an average of the times for the blood in the blood vessel 200 to return to the normal body temperature at rest when more than one resting transit time measurement (for example, three resting transit time measurements) was determined. The time for the blood in the blood vessel 200 to return to the normal body temperature with hyperemia may be determined using multiple temperature measurements from the guide wire sensor 222 with hyperemia. The time for the blood in the blood vessel 200 to return to the normal body temperature with hyperemia may be an average of the times for the blood in the blood vessel 200 to return to the normal body temperature with hyperemia when more than one hyperemic transit time measurement (for example, three hyperemic transit time measurements) was determined. The output of the thermodilution procedure may be a value for a mean transit time (Tmn) at maximal hyperemia. A coronary flow reserve (CFR) may moreover be determined by dividing a resting transit time measurement by a hyperemic transit time measurement.

At block 520, the process 500 can equalize the guide wire to the catheter. As discussed with respect to block 310 of the process 300, the one or more processors 112 can, for example, equalize a pressure measured by the guide wire 124, such as with the guide wire sensor 222, to a pressure measured by the catheter 122, such as with the catheter sensor 212. The catheter sensor 212 may be already equalized and zeroed to an atmospheric pressure prior to and at block 520 because, for instance, the catheter sensor 212 may be equalized and zeroed to the atmospheric pressure prior to the catheter being placed in the blood vessel 200. The equalizing can include one or more of (i) matching a mean pressure of a pressure waveform measured by the guide wire sensor 222 to a mean pressure of a pressure waveform measured by the catheter sensor 212, (ii) matching a timing of the pressure waveform measured by the guide wire sensor 222 to a timing of the pressure waveform measured by the catheter sensor 212, (iii) matching a gain of the pressure waveform measured by the guide wire sensor 222 to a level of the pressure waveform measured by the catheter sensor 212, (iv) matching an oscillator frequency of the pressure waveform measured by the guide wire sensor 222 to a frequency of the pressure waveform measured by the catheter sensor 212, or (v) matching a damping factor of the pressure waveform measured by the guide wire sensor 222 to a damping of the pressure waveform measured by the catheter sensor 212.

Block 520 may be performed when the shaft 210 and the wire 220 are positioned within the blood vessel 200 and the guide wire 124 is in the extended position relative to the catheter 122. If the guide wire 124 was in the extended position relative to the catheter 122 at block 510, the guide wire 124 may not have moved relative to the catheter 122 between block 510 and block 520. If the guide wire 124 was not in the extended position relative to the catheter 122 at block 510, the guide wire 124 can be moved to the extended position relative to the catheter 122 prior to block 520. Block 520 can be performed with hyperemia, such as while adenosine is infused. Such timing for performing equalization can result in the catheter sensor 212 and the guide wire sensor 222 equalizing in different pressure environments at least because the guide wire sensor 222 is separated from the catheter sensor 212 in the blood vessel 200 by the distance (for example, by at least 50, 60, 70, 75, 80, 90, 100, 110, 120, 125, 130,140, 150, 160, 170, 175, 180, 190, 200, 210, 220, 225, 230, 240, or 250 mm). This timing for equalization is counterintuitive because a clinician would not traditionally seek to equalize the guide wire sensor 222 to the catheter sensor 212 when the guide wire sensor 222 and the catheter sensor 212 are expected to be in different pressure environments. Such timing would be expected to result in a miscalibration of pressure measurements.

At block 530, the process 500 can determine pressure measurements with the guide wire and the catheter. For example, the one or more processors 112 can determine a pressure measurement at maximal hyperemia using the guide wire 124, such as with the guide wire sensor 222, and a pressure measurement at maximal hyperemia using the catheter 122, such as with the catheter sensor 212. The pressure measurements from the guide wire sensor 222 and the catheter sensor 212 at block 530 can each be a mean, median, mode, or other suitable value determined from a detected pressure waveform. The pressure measurements from the guide wire sensor 222 and the catheter sensor 212 may each be determined from a particular portion or an entirety of the detected pressure waveform for each heartbeat cycle. The detected pressure waveforms may be conditioned, filtered (such as to remove outliers), or processed prior to being used to determine the pressure measurements at block 530. Block 530 may be performed when the shaft 210 and the wire 220 are positioned within the blood vessel 200 and the guide wire 124 is in a retracted position relative to catheter 122, meaning the wire 220 is in the retracted position relative to the shaft 210. The guide wire 124 accordingly may be moved relative to the catheter 122 between block 520 and block 530 so that the guide wire 124 transitions from the extended position to the retracted position. Block 530 can be performed subsequent to equalizing at block 520 and without again equalizing the guide wire 124 to the catheter 122. Block 530 may be performed while the guide wire sensor 222 is in motion or once the guide wire sensor 222 is at rest, such as when the wire 220 is in a fully retracted position.

Block 530 may be performed within a short period of time, such as within 30, 20, 15, 10, 5, 4, 3, or 2 seconds (for example, within around 3-10 seconds when it is desired to minimize a delay between block 520 and block 530), subsequent to equalizing at block 520. Block 530 can be performed within the short time period at least because the wire 220 may be moved relatively quickly and safely from the extended position to the retracted position. In contrast, moving the wire 220 from the retracted position to the extended position may be a relatively slower, less safe, and more difficult operation to perform because the extending results in the wire 220 progressing farther into the blood vessel 200 generally along the middle of the blood vessel 200 and potentially traversing changes in path direction (including at least changes in tortuosity) and various obstacles (including at least the stenosis) without traumatizing the blood vessel 200 (for example, tearing the blood vessel 200 with a tip of the wire 220 or even causing the tip of the wire 220 to puncture and pass outside the blood vessel 200).

At block 540, the process 500 can generate an indication from the flow measurement and the pressure measurements. For example, the one or more processors 112 can generate an indication from the flow measurement at block 510 and the pressure measurements at block 530. The indication can be a value indicative of a resistance of the blood vessel 200 to a blood flow in the blood vessel 200 or a rate of blood flow, among other possible characteristics. The generating can, for instance, include determining the indication from a distal pressure Pd (sometimes referred to as true distal coronary pressure), which can equal an aortic pressure Pa less an absolute pressure gradient to the distal location where the guide wire sensor 222 was positioned when the guide wire 124 was in the extended position relative to the catheter 122. The distal pressure Pd can equal a difference between [i] the pressure measurement determined at block 530 with the catheter sensor 212 and [ii] a difference between the pressure measurement determined at block 530 with the guide wire sensor 222 and the pressure measurement determined at block 530 with the catheter sensor 212. The distal pressure Pd can be the pressure at maximal hyperemia because the constituent pressure measurements used to compute distal pressure Pd may have been measured at maximal hyperemia. The indication may be an index (similar to an IMR, CFR, an hyperemic microvascular resistance (HMR), or an invasive minimal microvascular resistance) that has a value determined by multiplying [i] the distal pressure Pd by [ii] the flow measurement at block 510 (for instance, the mean transit time Tmn at maximal hyperemia).

As background for some implementations of block 540 of the process 500, the computation of the flow measurement at block 510 may be derived as follows:

$$Resistance = \frac{Δ Pressure}{Flow} \qquad ( 1 )$$

where ΔPressure=Pd−Pv, Pd=distal pressure, Pv=venous pressure If Pv is assumed to be zero because Pv is likely very low, the resistance computation at block 540 may be simplified to:

$$Resistance = \frac{Pd}{Flow} \qquad ( 2 )$$

If

$$Flow ≅ \frac{1}{{T}_{mn}}$$

and Tₘₙ=mean transit time, the resistance computation may accordingly be adjusted to:

$$Resistance = Pd \times {T}_{mn} \qquad ( 3 )$$

At block 550, the process 500 can output an indication. For example, the one or more processors 112 can output the indication generated at block 540 for presentation to a user via the user interface 118, such as in the form of a numerical value, a plot, an alarm, or a coloring of an object that is presented to the user. The indication may permit the user to assess the blood vessel 200, such as the resistance of the blood vessel 200 to the blood flow in the blood vessel 200 or another characteristic of the blood vessel 200. The one or more processors 112 may additionally or alternatively output the indication for storage to the memory device 114, output the indication via wired or wireless communication to an electronic device, or output the indication to automatically activate one or more control operations (for example, to cause delivering of a fluid to the patient, determining a type or quantity of medication appropriate for the patient in view of the resistance of the blood vessel 200, ordering a medication for the patient, determining a size or type of stent for placement in the blood vessel 200 in view of the resistance of the blood vessel 200, placing a stent in the blood vessel 200, or submit a request for assistance from another clinician in view of the resistance of the blood vessel 200).

The process 500 can include one or more additional steps or may exclude one or more steps in some implementations. For example, the process 500 can include, after block 520 and prior to block 530, periodically (for instance, every 1 ms, 10 ms, or sufficiently frequently so as to appear instantaneous to a user) determining a pressure measurement with the guide wire 124 and outputting the pressure measurement as part of a pressure trace (sometimes referred to as a pullback trace) on a display to a user. The pressure trace may be used by the user to observe pressure changes in the blood vessel while the guide wire 124 retracts. The pressure trace can indicate to the user a location in the blood vessel where a relatively large change in pressure occurs and a stent may be placed to reduce that pressure change. In such an implementation, block 530 may be performed within around 30 seconds subsequent to equalizing at block 520. This duration may, for instance, be sufficient to determine the pressure measurements for the pressure trace, as well as generally still minimize a delay between block 530 and block 520. As another example, the process 500 can be performed together with the process 300 so that the indication output at block 550 may be output simultaneously or around the same time as the indication output at block 340 to provide more useful information to a clinician. This approach may moreover advantageously enable the clinician to obtain that useful information by performing fewer steps than may be traditionally required for obtaining two such indications, as a result at least of the clinician being able to perform a single series of movements with the catheter and the guide wire rather than repeating movements with the catheter and the guide wire to generate the two indications. As yet another example, although some generated indications or determined values are explained in connection with the process 500, other indications may be generated or values may be determined prior to initiating the process 500, during or between one or more steps of the process 500, or after completing the process 500, so as to further improve the efficiency of collecting useful information for a clinician when performing the process 500. Moreover, although certain examples of the process 500 are discussed in connection with hyperemia, the process may be performed without hyperemia in other examples.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing device can be a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements or states. Thus, such conditional language is not generally intended to imply that features, elements or states are in any way required for one or more embodiments or that one or more embodiments necessarily include logic for deciding, with or without author input or prompting, whether these features, elements or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Terms such as “substantially,” “about,” “approximately” or the like as used in referring to a relationship between two objects is intended to reflect not only an exact relationship but also variances in that relationship that may be due to various factors such as the effects of environmental conditions, common error tolerances, manufacturing variances, or the like. It should further be understood that although some values or other relationships may be expressed herein without a modifier, these values or other relationships may also be exact or may include a degree of variation due to various factors such as the effects of environmental conditions, common error tolerances, or the like.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others.

What is claimed is:

1. A method of assessing a blood vessel of a patient, the method comprising:
equalizing, by one or more processors, a pressure measured by a guide wire sensor to a pressure measured by a catheter sensor when the guide wire sensor is positioned within the blood vessel and downstream from a stenosis of the blood vessel relative to a blood flow in the blood vessel and when the catheter sensor is positioned within the blood vessel and upstream from the stenosis relative to the blood flow in the blood vessel;
subsequent to said equalizing the pressure measured by the guide wire sensor to the pressure measured by the catheter sensor, determining, by the one or more processors, a pressure measurement from the guide wire sensor when the guide wire sensor is positioned upstream from the stenosis relative to the blood flow in the blood vessel;
generating, by the one or more processors, an indication responsive to the pressure measurement from the guide wire sensor when the guide wire sensor was positioned upstream from the stenosis relative to the blood flow in the blood vessel; and
outputting, by the one or more processors, the indication for presentation to a user.

2. The method of claim 1, wherein said equalizing comprises matching the pressure measured by the guide wire sensor to the pressure measured by the catheter sensor.

3. The method of claim 1, wherein said equalizing comprises matching a mean pressure of a pressure waveform measured by the guide wire sensor to a mean pressure of a pressure waveform measured by the catheter sensor.

4. The method of claim 3, wherein said equalizing comprises (i) matching a timing of the pressure waveform measured by the guide wire sensor to a timing of the pressure waveform measured by the catheter sensor and (ii) matching a gain of the pressure waveform measured by the guide wire sensor to a level of the pressure waveform measured by the catheter sensor.

5. The method of claim 1, wherein said equalizing is performed responsive to an input from the user.

6. The method of claim 1, wherein said generating comprises determining a pressure ratio using the pressure measurement from the guide wire sensor when the guide wire sensor was positioned upstream from the stenosis relative to the blood flow in the blood vessel.

7. The method of claim 6, wherein the pressure ratio equals (i) the pressure measurement from the guide wire sensor when the guide wire sensor was positioned upstream from the stenosis relative to the blood flow in the blood vessel, divided by (ii) a pressure measurement from the catheter sensor when the catheter sensor was positioned upstream from the stenosis relative to the blood flow in the blood vessel.

8. The method of claim 1, wherein said determining is performed within 5 seconds of said equalizing.

9. The method of claim 1, wherein the stenosis comprises a coronary lesion.

10. The method of claim 1, further comprising generating a pressure waveform with a transducer of the guide wire sensor.

11. A system for assessing a blood vessel of a patient, the system comprising:
one or more processors programmed to:
equalize a pressure measured by a guide wire sensor to a pressure measured by a catheter sensor when the guide wire sensor is positioned within the blood vessel and downstream from a stenosis of the blood vessel relative to a blood flow in the blood vessel and when the catheter sensor is positioned within the blood vessel and upstream from the stenosis relative to the blood flow in the blood vessel,
subsequent to equalizing the pressure measured by the guide wire sensor to the pressure measured by the catheter sensor, determine a pressure measurement from the guide wire sensor when the guide wire sensor is positioned upstream from the stenosis relative to the blood flow in the blood vessel,
generate an indication responsive to the pressure measurement from the guide wire sensor when the guide wire sensor was positioned upstream from the stenosis relative to the blood flow in the blood vessel, and
output the indication for presentation to a user; and
a memory device in communication with the one or more processors, the memory device being configured to store the indication.

12. The system of claim 11, wherein the one or more processors are programmed to equalize the pressure measured by the guide wire sensor to the pressure measured by the catheter sensor by matching the pressure measured by the guide wire sensor to the pressure measured by the catheter sensor.

13. The system of claim 11, wherein the one or more processors are programmed to equalize the pressure measured by the guide wire sensor to the pressure measured by the catheter sensor by matching a mean pressure of a pressure waveform measured by the guide wire sensor to a mean pressure of a pressure waveform measured by the catheter sensor.

14. The system of claim 13, wherein the one or more processors are programmed to equalize the pressure measured by the guide wire sensor to the pressure measured by the catheter sensor by (i) matching a timing of the pressure waveform measured by the guide wire sensor to a timing of the pressure waveform measured by the catheter sensor and (ii) matching a gain of the pressure waveform measured by the guide wire sensor to a level of the pressure waveform measured by the catheter sensor.

15. The system of claim 11, wherein the one or more processors are programmed to equalize the pressure measured by the guide wire sensor to the pressure measured by the catheter sensor responsive to an input from the user.

16. The system of claim 11, wherein the indication comprises a pressure ratio determined from the pressure measurement from the guide wire sensor when the guide wire sensor was positioned upstream from the stenosis relative to the blood flow in the blood vessel.

17. The system of claim 16, wherein the pressure ratio equals (i) the pressure measurement from the guide wire sensor when the guide wire sensor was positioned upstream from the stenosis relative to the blood flow in the blood vessel, divided by (ii) a pressure measurement from the catheter sensor when the catheter sensor was positioned upstream from the stenosis relative to the blood flow in the blood vessel.

18. The system of claim 11, wherein the one or more processors are programmed to, within 30 seconds of equalizing the pressure measured by the guide wire sensor to the pressure measured by the catheter sensor, determine the pressure measurement from the guide wire sensor when the guide wire sensor was positioned upstream from the stenosis relative to the blood flow.

19. The system of claim 11, wherein the stenosis comprises a coronary lesion.

20. The system of claim 11, further comprising a guide wire and a catheter, the guide wire sensor comprising a transducer and being attached to the guide wire, the catheter comprising the catheter sensor.

21. A method of assessing a blood vessel of a patient, the method comprising:
equalizing, by one or more processors, a pressure measured by a guide wire sensor to a pressure measured by a catheter sensor when a guide wire comprising the guide wire sensor is positioned within a blood vessel and in an extended position relative to a catheter comprising the catheter sensor;
subsequent to said equalizing the pressure measured by the guide wire sensor to the pressure measured by the catheter sensor:
determining, by the one or more processors, a first pressure measurement from the guide wire sensor when the guide wire is positioned within the blood vessel and in a retracted position relative to the catheter, and
determining, by the one or more processors, a second pressure measurement from the catheter sensor when the guide wire is positioned within the blood vessel and in the retracted position relative to the catheter;
generating, by the one or more processors, an indication responsive to the first pressure measurement and the second pressure measurement; and
outputting, by the one or more processors, the indication for presentation to a user.

22. The method of claim 21, wherein said equalizing comprises matching a mean pressure of a pressure waveform measured by the guide wire sensor to a mean pressure of a pressure waveform measured by the catheter sensor.

23. The method of claim 21, wherein the indication reflects a resistance of the blood vessel to a blood flow in the blood vessel.

24. The method of claim 21, wherein said generating comprises generating the indication responsive to a comparison of the first pressure measurement and the second pressure measurement.

25. The method of claim 21, wherein said generating comprises generating the indication responsive to a difference between (i) the second pressure measurement and (ii) a difference between the first pressure measurement and the second pressure measurement.

26. The method of claim 21, further comprising determining, by the one or more processors, a flow measurement of a blood flow in the blood vessel, wherein said generating comprises generating the indication further responsive to the flow measurement.

27. The method of claim 26, further comprising determining, by the one or more processors, the flow measurement using a plurality of temperature measurements from the guide wire sensor.

28. The method of claim 26, further comprising determining, by the one or more processors, the flow measurement from a resting transit time measurement and a hyperemic transit time measurement.

29. The method of claim 21, wherein said determining the second pressure measurement is performed within 10 seconds of said equalizing.

30. A system for assessing a blood vessel of a patient, the system comprising:
one or more processors programmed to:
equalize a pressure measured by a guide wire sensor to a pressure measured by a catheter sensor when a guide wire comprising the guide wire sensor is positioned within a blood vessel and in an extended position relative to a catheter comprising the catheter sensor,
subsequent to equalizing the pressure measured by the guide wire sensor to the pressure measured by the catheter sensor:
determine a first pressure measurement from the guide wire sensor when the guide wire is positioned within the blood vessel and in a retracted position relative to the catheter, and
determine a second pressure measurement from the guide wire sensor when the guide wire is positioned within the blood vessel and in the retracted position relative to the catheter,
generate an indication responsive to the first pressure measurement and the second pressure measurement, and
output the indication for presentation to a user; and
a memory device in communication with the one or more processors, the memory device being configured to store the indication.

31. The system of claim 30, wherein the one or more processors are programmed to equalize the pressure measured by the guide wire sensor to the pressure measured by the catheter sensor by matching the pressure measured by the guide wire sensor to the pressure measured by the catheter sensor.

32. The system of claim 30, wherein the one or more processors are programmed to equalize the pressure measured by the guide wire sensor to the pressure measured by the catheter sensor by (i) matching a mean pressure of a pressure waveform measured by the guide wire sensor to a mean pressure of a pressure waveform measured by the catheter sensor, (ii) matching a timing of the pressure waveform measured by the guide wire sensor to a timing of the pressure waveform measured by the catheter sensor, and (ii) matching a gain of the pressure waveform measured by the guide wire sensor to a level of the pressure waveform measured by the catheter sensor.

33. The system of claim 30, wherein the indication reflects a resistance of the blood vessel.

34. The system of claim 30, wherein the one or more processors are programmed to generate the indication responsive to a comparison of the first pressure measurement and the second pressure measurement.

35. The system of claim 30, wherein the one or more processors are programmed to generate the indication responsive to a difference between (i) the second pressure measurement and (ii) a difference between the first pressure measurement and the second pressure measurement.

36. The system of claim 30, wherein the one or more processors are programmed to:
determine a flow measurement of a blood flow in the blood vessel; and
generate the indication further responsive to the flow measurement.

37. The system of claim 36, wherein the one or more processors are programmed to determine the flow measurement using a plurality of temperature measurements from the guide wire sensor.

38. The system of claim 36, wherein the one or more processors are programmed to determine the flow measurement from a resting transit time measurement and a hyperemic transit time measurement.

39. The system of claim 36, wherein the one or more processors are programmed to determine the flow measurement from a plurality of resting transit time measurements and a plurality of hyperemic transit time measurements.

40. The system of claim 30, wherein the one or more processors are programmed to determine the second pressure measurement within 30 seconds of equalizing the pressure measured by the guide wire sensor to the pressure measured by the catheter sensor.
